(19) United States
(12) Patent Application Publication
(10) Pub. No.: US 2014/0257149 A1
(43) Pub. Date: Sep. 11, 2014

(54) Skin Rejuvenation Therapy Machine

(71) Applicant: WAYNE GEORGE COTTON, CARIGNAN (CA)

(72) Inventor: WAYNE GEORGE COTTON, CARIGNAN (CA)

(21) Appl. No.: 13/794,048

(22) Filed: Mar. 11, 2013

(51) Int. Cl. A61H 23/04; A61F 7/00

(52) U.S. Cl. CPC A61H 23/04; A61F 7/0085
USPC 601/18; 601/46

(57) ABSTRACT

An apparatus comprises several different body part wraps that connect individually to two fluidic systems. The apparatus reservoirs, hot and cold, operate with pumps. The body part wraps have two separate compartments for hot and cold fluid. The body part wraps allow hot fluid to be pumped into the first compartment and exit back to the reservoir. Then the cold fluid enters the second compartment and exits. The pumps push the fluids through the body part wraps creating a vibrating massage and activates the body's acupressure points. The apparatus provides a therapy by using two fluids, vibrating massage and the body's acupressure points to create a method to facilitate blood flow by stimulating circulation, thus speeding up the body's natural self-curative abilities.

The skin rejuvenation apparatus provides unequalled benefits by a series of regular treatments. The method facilitates blood flow by stimulating circulation inside the energy pathways of the human body. Blood flow travels through the meridian lines and provides all of the body's nutritive elements and energy to all cells and muscles, thus accelerating the rejuvenation of all skin cells. This apparatus comprises several different types of body wraps to be used individually. The body part wraps are constructed with four layers of clear flexible vinyl. The four pieces of clear flexible vinyl are RF welded making one specific body part wrap with two chambers. The first chamber is to allow hot fluid to enter and continuously flow throughout the body part wrap and exit. This process is executed for a determined length of time to initiate vasodilation which contributes to oxygenation and nourishment of the skin cells. The second chamber is to allow cold fluid to enter and continuously flow throughout the body wrap part and exit. This process is executed for a determined length of time to activate the body's natural repair mechanism and the removal of toxins. The clear flexible body part wraps are manufactured with acupressure points which activate and stimulate the body's acupuncture points. This process promotes a significant production of collagen, playing a determining role in our skin's appearance. The body's acupressure points improve overall health and well-being to the body's skin.

The apparatus consists of two fluidic systems. Both systems have individual temperature controlled fluidic reservoirs to keep the hot and cold at their exact required temperatures. When in operation, both systems have each an individual pump that stimulates a pulsation massage within the flexible body part wrap. The fluidic systems always operate individually one after the other. The sequence is hot fluid for seven minutes followed by cold fluid for three minutes. This process is executed for 6 times to create one hour of skin rejuvenation therapy. This present invention provides countless advantages in the overall improvement of blood flow throughout the body and overall skin appearances such as the reduction of wrinkles, cellulite, acne, shrinkage of pore size and skin softness.

### BACKGROUND OF THE INVENTION

This invention is drawn towards the common knowledge of how our bodies function when it pertains to repairing itself. Cicatrisation is an intricate process in which the skin repairs itself after injury. In normal skin, the epidermis (outermost layer) and the dermis (inner layer) exist in a steady—state equilibrium, forming a protective barrier against the external environment. Once the protective barrier is broken, the process of wound healing is immediately set in motion. Upon injury to the skin, a set of complex biochemical events takes place in a closely cascade to repair the injury. Within seconds the body's blood is present to do all the necessary functions to repair the body's skin. The chief function of blood is to bring oxygen and nutrients to tissues in the body, and carry away carbon dioxide, lactic acid, and other waste material. This waste is flushed out of the body in urine, sweat and lungs. (References: pages 26,27,28,29,30,31,32,33,34,35)

This invention is drawn towards the common knowledge how moist hear is effective at warming tissue. Heat creates higher tissue temperatures, which produces vasodilation that increases the blood flow, a surplus of nutrients, increase supply of oxygen and the elimination of carbon dioxide and metabolic waste. Increasing the blood flow accelerates the rehabilitation for fast healing. (References: pages 12,13,14)

This invention is drawn towards the common knowledge how cold therapy is effective in contracting blood vessels on the skins surface. At the same time, blood rushes to the body's core to keep the vital organs at a constant temperature. Then, when the cold therapy ends, the blood capillaries expand to up to four times their normal size as the blood rushes back to the skin surface. (Reference 13,14,15,16,17,18,19)

This invention is drawn towards the common knowledge how acupressure is an ancient healing art using pressure to key healing points which stimulate the body's natural self-curative abilities. Acupressure releases tension, increases circulation and develops vibrant overall health. When the acupressure points in our body are activated by pressure and massage, this helps promote and increase blood flow throughout the skin, while helping remove toxins. Acupressure points have a high electrical conductivity at the surface of the skin, thus conduct and channel healing energy most effectively. Acupressure points are the most potent healing energy used. (References: pages 20,23,24,25)

This invention is drawn to the common knowledge of vibration therapy. When using low vibration therapy the vibrations widen the capillary vessels improving on microcirculation, lymphatic drainage and boost tissue metabolism. Vibration massage therapy has a direct impact on the skin. The friction increases body temperature and causes dilation of blood vessels, which in return allow rich nutrient blood to reach the surface increasing the condition of the skin. (References: pages 21,22)

### BRIEF SUMMARY OF THE INVENTION

The general idea of the invention is that this method enhances the body's natural mechanisms that repair the body's skin. The apparatus uses hot fluid to heat tissue, vibration and massage to create friction that heats tissue and activates the acupressure points all to enhance and increase blood flow. The apparatus uses cold to contract the body's blood vessels, but when the cold process is terminated, the blood vessels can grow four times their normal size and that allows a surplus of blood rush back to the surface of the body. The human body's blood is the source of all nutrients and oxygen that repair the body's skin. The apparatus uses these five common knowledge's or therapies as an advantage by applying as one new therapy. The apparatuses method is a therapy and its advantages is that it facilitates blood flow by stimulating circulation and thus enhancing rejuvenation of all skin cells. The present invention uses body wraps that have two compartments and each compartment is connected to a fluidic system. The first fluidic system is a heat reservoir and the second fluidic system is a cold reservoir. Both fluidic systems have their own individual pumps that create a flow of hot or cold fluid throughout the body part wrap and as it flows through the body part wrap, it exits back to their designated reservoirs. The pump creates a vibrating flow that also creates a massage effect in the body part wrap. This effect is the method of stimulating friction and the acupressure points in the human body. The fluidic systems are temperature controlled with individual electronic thermostats keeping the fluid within plus or minus 0.05 degrees of their pre-set temperature. The reservoirs both operate individually with thermoelectric techs, one to heat the fluid in the hot reservoir and one to cool the fluid in the cold reservoir. To operate the apparatus in harmony, it uses an automat to control all mechanical functions.

A primary objective of the present invention is it provides an apparatus and method of use, that provides advantages not taught in the prior arts.

Another objective is it provides an invention capable of enhancing blood flow in the human body.

A further objective is it provides and accelerates the body's repair mechanisms.

A still further objective is to provide such and invention capable to apply this method to all parts of the human body using the inventions flexible body part wraps.

Other features and advantages of the present invention will become apparent from the following more detailed description, taken in conjunction with the accompanying drawings, which illustrate, by the way of example, the principles of the invention.

### BRIEF DESCRIPTION OF THE DRAWINGS

The accompanying drawings illustrate the present invention. In such drawings:

FIG. 1A, is a view of two pieces of clear flexible vinyl RF welded to create the hot compartment.

FIG. 1B, is a view of the two pieces of clear flexible vinyl RF welded to create the cold compartment.

FIG. 1C, is a view of FIG. 1A and FIG. 1B RF welded together to make the complete body part wrap with a hot and cold compartments.

FIG. 2, is a complete view of a functional body wrap part.

FIG. 3, is a view of both fluidic systems, showing the flow of both hot and cold systems throughout the body part wrap.

### DETAILED DESCRIPTION OF THE INVENTION

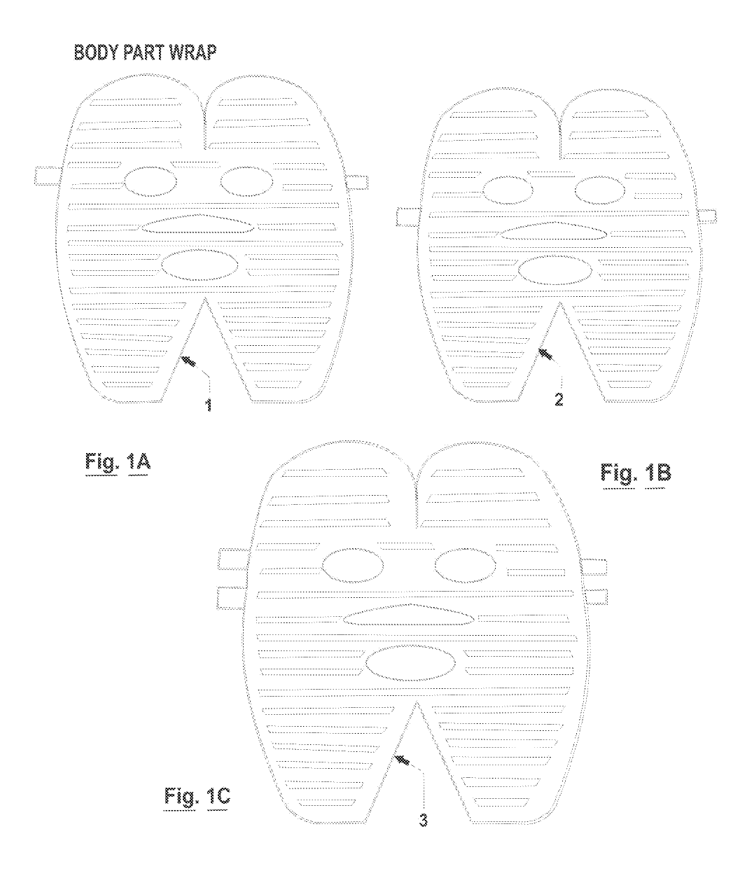

In FIG. 1A, there are two pieces of clear flexible vinyl RF welded together to make one compartment. It is to show that it will be used for the hot fluid. In FIG. 1B, there are two pieces of clear flexible vinyl RF welded together to make another compartment. It is to show it will be used for the cold fluid. FIG. 1C shows FIG. 1A and FIG. 1B, RF welded together to make a complete body wrap part with two components, one for the hot fluid and one for the cold fluid.

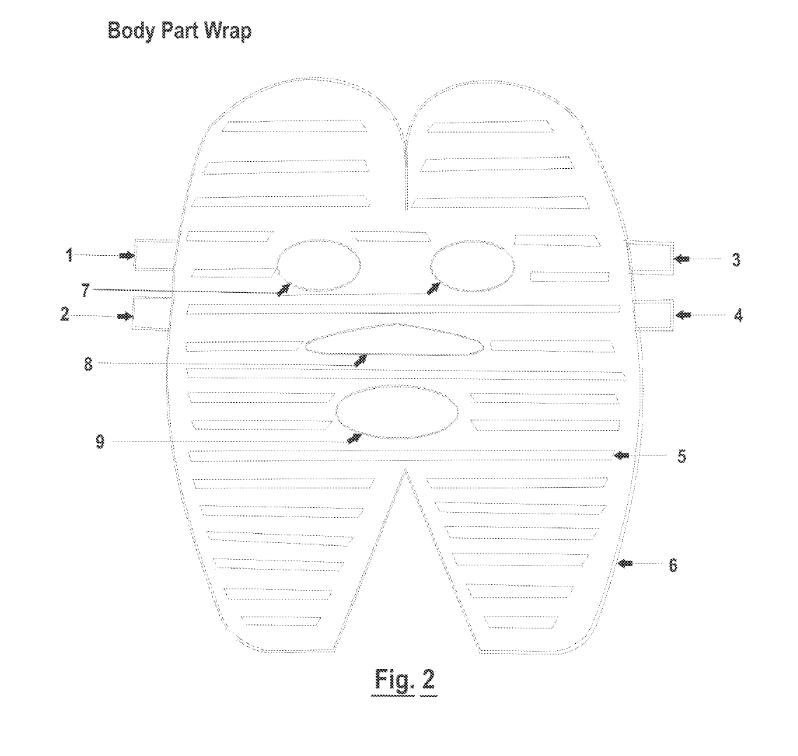

FIG. 2, shows a complete body wrap part and its primary functions. FIG. 2, portion 1, shows the entry point where hot fluid enters. Portion 3, shows where the hot fluid is to exit. In FIG. 2, portion 2, shows the entry point where cold fluid enters. Portion 4 shows where the cold fluid is to exit. In FIG. 2, portion 5, shows one of several RF welded bars that are there for the primary reason, that flexible body wrap parts when filled with either hot or cold fluid, will not balloon and remain flat. In FIG. 2, portion 6, is a double RF weld all around the body part wrap. This is to secure the body wrap from possible leakage. In FIG. 2, portions 7, 8 and 9 are the portions that have a double RF weld and cut outs for the eyes, nose and mouth to function.

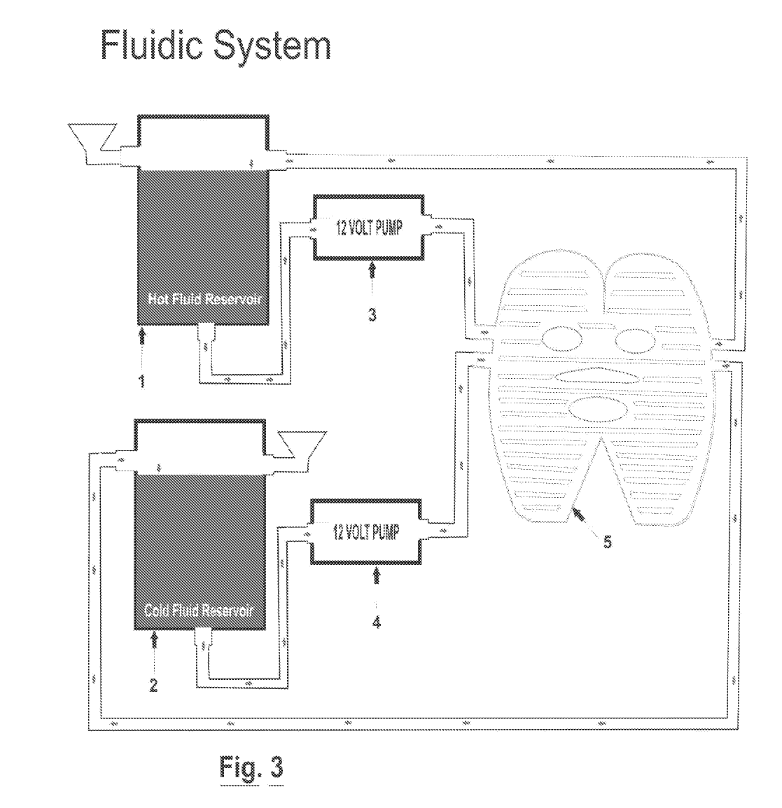

FIG. 3, shows the complete function and process for the skin rejuvenation therapy. FIG. 3, portion 1, is the hot reservoir that is heated with a thermoelectric tech. This fluid is heated until it reaches 105 degrees Fahrenheit and the temperature is maintained by a thermostat within plus or minus 0.05 degrees. FIG. 3, portion 3, is a 12 volt pump and this pump draws from the hot reservoir to push hot fluid into portion 5, the body wrap part using the first one of its compartments. In this process fluid enters and exits returning to the reservoir continuously for seven minute. When the pump pushes the fluid throughout the body wrap it makes it vibrate giving massage effects that activate and increase the blood flow bringing more blood to the body part area being treated. This surplus of blood with its nutrients and oxygen, contribute to the skins rejuvenation. FIG. 3, portion 2, is the cold reservoir that is chilled with a thermoelectric tech. This fluid is chilled to 48 degrees Fahrenheit and the temperature is maintained by the thermostat within plus or minus 0.05 degrees. FIG. 3, portion 4, is a 12 volt pump and the pump draws for the cold reservoir to push cold fluid into portion 5, the body part wrap using the second compartment. In this process fluid enters and exits continuously for three minutes. When the pump pushes the fluid throughout the body wrap it makes it vibrate giving the massage effects that activate the acupressure points.

The cold fluids chilling effect contracts the blood vessels, but once the process is terminated the blood vessels expand up to four times their normal size thus allowing a large surplus of blood to enter the area being treated. When the cold process terminates the hot process starts again. The Ten minute process is repeated six times in one hour to do a complete one skin rejuvenation therapy session.

1) What I claim as my invention is using five common known facts that; heat therapy, acupressure therapy, low vibration therapy, cold therapy and the bodies skin repair mechanisms, and apply them all together as one new method or therapy: This new therapy enhances and increases the body's blood flow to speed up the rejuvenation of all damaged skin cells being treated.

2) What I claim as my invention is an apparatus comprising of several different body wraps made of four pieces of vinyl each, having two compartments, one for cold fluid and the other for hot fluid, with an entry port and exit port to accommodate a hot fluid and a cold fluid to flow separately in their own compartments.

3) What I claim as my invention is by using 12 volt pumps to push fluid through the body part wrap rather than draw or pull it through, creates a vibrating massage to activate the body's acupressure points enhancing blood circulation.
